(19) United States
(12) Patent Application Publication
(10) Pub. No.: US 2013/0225879 A1
(43) Pub. Date: Aug. 29, 2013

(54) PROCESS FOR THE SELECTIVE PREPARATION OF 1-PROPANOL, ISO-BUTANOL AND OTHER C3+ ALCOHOLS FROM SYNTHESIS GAS AND METHANOL

(71) Applicants: Burcin Temel, Hellerup (DK); Poul Erik Højlund Nielsen, Fredensdorg (DK); Pablo Beato, Copenhagen (DK)

(72) Inventors: Burcin Temel, Hellerup (DK); Poul Erik Højlund Nielsen, Fredensdorg (DK); Pablo Beato, Copenhagen (DK)

(21) Appl. No.: 13/883,059

(22) PCT Filed: Nov. 8, 2010
(86) PCT No.: PCT/EP2010/006780

§ 371 Date: May 2, 2013

(51) Int. Cl. C07C 29/32

(52) U.S. Cl. CPC C07C 29/32
USPC 568/903

(57) ABSTRACT

Process for the preparation of a product alcohol mixture comprising the steps of: (a) providing a synthesis gas comprising carbon monoxide and hydrogen (b) providing an amount of methanol and a second source alcohol Rₙ—CH₂—CH₂—OH comprising n+2 carbon atoms (Rₙ═CₙH₂ₙ+₁, n≧O) to the synthesis gas to obtain a selective alcohol synthesis mixture (c) converting the selective synthesis mixture in presence of one or more catalysts catalysing the conversion of the synthesis gas mixture into a product alcohol mixture in which the initially dominating alcohol is a preferred Cₙ₊₃ alcohol having the structure Rₙ—CH(CH₃)—CH₂—OH (d) withdrawing the product alcohol mixture of step (c).

The present invention relates to the production of C3+ alcohols. In particular, the invention is a process for the preparation of these alcohols by conversion of carbon monoxide and hydrogen containing synthesis gas admixed with one or more source alcohols in presence of a catalyst containing oxides of copper, zinc and aluminium.

It is known that higher alcohols and other oxygenates are formed as by-products in the catalytic methanol synthesis from synthesis gas.

It is also known that higher alcohol products can be produced directly from synthesis gas.

US patent application No. 2009/0018371 discloses a method for the producing alcohols from synthesis gas without presenting experimental data. The synthesis gas is in a first step partially converted to methanol in presence of a first catalyst and in a second step methanol is converted with a second amount of synthesis gas to a product comprising C₂-C₄ alcohols in presence of a second catalyst. The second amount of synthesis gas can include unreacted synthesis gas from the first step. In this application, the product composition is proposed to be controlled by controlling the H₂/CO ratio.

Smith and Anderson (J. Catal. 85, 428-436, 1984) present a chain growth scheme for alcohols formed from synthesis gas, and preliminary experiments involving production of higher alcohols from a single lower alcohol and synthesis gas. The experiments presented only demonstrate a rate of production of higher alcohols in the range 1-33 g/kg/h.

Furthermore methods of producing methanol and higher alcohols at reaction temperatures between 360° C. and 440° C. have been disclosed in U.S. Pat. No. 4,513,100. This disclosure does not discuss the specificity of the production of higher alcohols.

It is therefore an object of the present invention to provide a process for providing from synthesis gas a product alcohol mixture comprising higher alcohols.

It is a further object of the present disclosure to provide an improved means of controlling the composition of the product alcohol mixture.

The alcohol synthesis requires a high concentration of carbon monoxide in the synthesis gas. A useful synthesis gas has a H₂/CO ratio between 0.3 and 3, but the range from 0.5 to 2 or even 0.5 to 1 is preferred, since a lower H₂/CO ratio will reduce the reaction rate. The synthesis gas for the higher alcohol synthesis may be prepared by the well known steam reforming of liquid or gaseous hydrocarbons or by means of gasification of carbonaceous material, preferably a carbonaceous material having a high C/H ratio such as coal, heavy oil or bio mass resulting in a synthesis gas rich in CO.

When using a promoted copper catalyst, such as an alcohol formation catalyst together with a synthesis gas having a high content of carbon monoxide, it is well known to the person skilled in the art that the catalyst has a relatively short operation time. The catalyst bed will after a time on stream be clogged with waxy material and has to be removed.

We have found that this problem arises during preparation of the synthesis gas under conditions providing a relatively high content of carbon monoxide, such as H₂/CO below 1.0. Carbon monoxide reacts with the steel equipment used in the synthesis gas preparation and forms i.e. iron and nickel carbonyl compounds. When transferred to the oxidic alcohol formation catalyst, these compounds catalyse the Fischer-Tropsch polymerisation of CO with H₂ to higher paraffins and a waxy material is formed on the catalyst, resulting in catalyst clogging and deactivation.

By avoiding the presence of metal carbonyl compounds in the synthesis gas upstream of the alcohol synthesis, e.g. according to a method as described in the unpublished Danish patent application PA201000591, the operation time of the catalyst can be much improved. Other methods of avoiding the presence of metal carbonyl compounds may involve the use of materials which do not contribute to formation of metal carbonyls such as glass or ceramics.

It has further been found that addition of methanol, and in particular alcohols higher than methanol to the synthesis gas, results in an increase in the yield of higher alcohols when compared to the known methanol synthesis gas mixture.

The addition of methanol and a second source alcohol has even further been found to provide a means of controlling the composition of the product alcohol mixture of generated by the process. Such alcohols added to the synthesis gas are referred to as source alcohols.

Pursuant to the above findings, this invention is a process for the preparation higher alcohols such as ethanol, 1-propanol, 2-propanol, butanols, pentanols and hexanols, which in its broadest embodiment comprises the steps of:

(a) providing a synthesis gas comprising carbon monoxide and hydrogen,

(b) providing an amount of methanol and a second source alcohol Rₙ—CH₂—CH₂—OH comprising n+2 carbon atoms (Rₙ═CₙH₂ₙ₊₁, n≧0) to the synthesis gas to obtain a selective alcohol synthesis mixture,

(c) converting the selective synthesis mixture in presence of one or more catalysts catalysing the conversion of the synthesis gas mixture into a product alcohol mixture in which the initially dominating alcohol is a preferred Cₙ₊₃ alcohol having the structure Rₙ—CH(CH₃)—CH₂—OH,

(d) withdrawing the product of step (c).

Specifically the second source alcohol may be ethanol with the preferred alcohol being 1-propanol, or the second source alcohol may be 1-propanol with the preferred alcohol is 2-methyl-1-propanol.

In embodiments of the invention methanol is present in a concentration corresponding within ±10% from the equilibrium at reaction temperature and the second source alcohol concentration in the selective alcohol synthesis mixture is 0.1-60 volume %. The second source alcohol concentration shall preferably be from 0.5 to 2.0 times the concentration of methanol.

In a preferred embodiment, the preferred alcohol is present in higher concentration than any of the alcohols being isomers to the preferred alcohol.

In another preferred embodiment the one or more catalysts in step (c) comprise either copper on a support or copper and optionally one or both of zinc oxide and aluminium oxide and may optionally be promoted with one or more metals selected from alkali metals, basic oxides of earth alkali metals and lanthanides, and in which the copper may be provided as metallic copper or copper oxide.

In yet another preferred embodiment the metal carbonyl compounds are substantially absent from the selective synthesis mixture, such as having a concentration of 0-10 ppbw (parts per billion by weight, i.e. 10⁻⁹ g/g) or preferably 0-2 ppbw. In a further preferred embodiment this absence may be obtained by removing the metal carbonyl compounds from the synthesis gas or the selective synthesis mixture by contacting the synthesis gas or the selective synthesis mixture with a sorbent. This sorbent may in a further embodiment be arranged on top of a fixed bed of the one or more catalysts catalysing the conversion of the synthesis gas mixture.

In a preferred embodiment the conversion of the synthesis gas mixture may be performed at a pressure of between 2 and 15 MPa and a temperature of between 270° C. and 330° C.

The process may, in a further preferred embodiment comprise the further steps of

(d) cooling the withdrawn product in step (c); and

(e) contacting the cooled product with a hydrogenation catalyst, such as a temperature of between 20° C. and 200° C.

In further embodiments the hydrogenation catalyst comprises copper and optionally one or both of zinc oxide and aluminium oxide or as an alternative, the hydrogenation catalyst may comprise platinum and/or palladium.

In a further preferred embodiment all or a part of the product alcohol mixture recycled, optionally after being passed through a separation such as a distillation step, wherein a separation may take place according to one or more of branching and chain length.

In a specific embodiment the product alcohol mixture is used combination with recycling provides the possibility to partially or fully recycle only short linear alcohols, such as those comprising no more than 3 carbon atoms (methanol, ethanol and propanol) for using these as source alcohols forming desirable product alcohols.

Separation in combination with recycling also provides the possibility to partially or fully withdraw branched alcohols, as they are end-products, which cannot contribute further to the synthesis of higher alcohols.

In a further embodiment, the process may further comprise the process step (f) of withdrawal of alcohols above having more than 4 carbon atoms, and recycling of alcohols below having less than 3 carbon atoms.

As used hereinbefore and in the following description and the claims, the terms “higher alcohols” and C₃+ alcohols refer to alcohols having at least 3 carbon atoms.

Similarly, as used hereinbefore and in the following description and the claims, the term “source alcohols” refers to alcohols being supplied with the synthesis gas to the oxidic catalyst.

As used hereinbefore and in the following description and the claims, the mixture of synthesis gas and source alcohols is called the specific alcohol synthesis mixture.

As used hereinbefore and in the following description and the claims, the term “product alcohol mixture” refers to the mixture of alcohols downstream the catalyst. The product alcohol mixture will be a complex mixture of alcohols, due to the fact that the same source alcohols may undergo side reactions to form different product alcohols, and due to the fact that the product alcohols may also react by similar reactions forming higher product alcohols.

The term selective in relation to a chemical reaction in the following will refer to a preferred or dominating product of the reaction not an absolute selectivity. Therefore, significant amounts of by-products e.g. other alcohols will be present.

The term “product alcohol mixture” is not necessarily intended to refer to an actual mixture in the process, in that it may be referred to as if it does not include e.g. the remaining source alcohols and synthesis gas fed to the process. The product alcohol mixture will comprise by-products of the reaction.

The terms “dominated by” and “initially dominated by” a specific alcohol in relation to the product alcohol mixture shall be taken to cover that this specific alcohol will be present in the highest concentration not considering the source alcohols and after a reaction time where the higher alcohols formed have not reacted to a significant extent. In practice the term dominating product alcohol may define either the product alcohol having the highest concentration, or the product alcohol having the highest concentration among isomers.

The chemical nomenclature used hereinbefore and in the following description and the claims uses the formula Rₙ—CH₂—CH₂—OH comprising n+2 carbon atoms (n≧0) as a general second source alcohol including ethanol, in which case Rₙ simply is a hydrogen atom. In the general case Rₙ may indicate any alkyl group such as Rₙ═CₙH₂ₙ₊₁. In accordance with this the preferred (product) alcohol is indicated by the structure Rₙ—CH(CH₃)—CH₂—OH, where Rₙ also may be a hydrogen atom or an alkyl group.

Catalysts being active in the conversion of synthesis gas to higher alcohols are per se known in the art. For use in the present invention a catalyst comprise either copper on a support or copper and optionally one or both of zinc oxide and aluminium oxide and may optionally be promoted with one or more metals selected from alkali metals, basic oxides of earth alkali metals and lanthanides. A preferred catalyst consists of copper, zinc oxide and aluminium oxide and optionally promoted with one or more metals selected from alkali metals, basic oxides of earth alkali metals and lanthanides being commercially available from Haldor Topsøe A/S, Denmark.

The invention relates to selective formation of higher alcohols. Specific embodiments include the formation of 1-propanol by combining methanol and ethanol, and the formation of 2-methyl-1-propanol by combining methanol and 1-propanol.

As already discussed above, a preferred embodiment involves the absence from the synthesis gas and the selective synthesis mixture of metal carbonyl compounds, in particular iron and nickel carbonyls, in order to prevent formation of waxy material on the alcohol preparation catalyst due to the Fischer-Tropsch reaction catalysed by metal carbonyl compounds being otherwise present in the synthesis gas. This absence of metal carbonyl compounds may be attained either by an appropriate selection of materials for process equipment, or it may be attained by use of a sorbent.

Wax formation may also be avoided by other methods of avoiding the presence of metal carbonyl compounds, which may involve the use of materials which do not contribute to formation of metal carbonyls, such as glass or ceramics. Finally, it may also be found acceptable to allow presence of metal carbonyls, at the cost of a shorter lifetime of the oxidic alcohol formation catalyst.

The disclosure involves the addition of two alcohols to the synthesis gas upstream the alcohol reactor in order to increase the production yield of desired higher alcohols. Addition of methanol only, slightly improves the formation of higher alcohols. Furthermore, methanol formation from synthesis gas is an exothermic process and a methanol content in the gas below the equilibrium value for the temperature, and will give rise to a drastic temperature increase at reactor inlet due to a rapid methanol formation. Thus, by adding methanol in an amount, which adjusts the reaction mixture towards the thermodynamic equilibrium with respect to the content of methanol in the synthesis reaction, exothermal methanol formation will be avoided or reduced at the reactor inlet with the beneficial effect that the temperature of the reactor may be controlled better.

A further effect of adding methanol is, without being bound by theory, assumed to be that the presence of methanol has a kinetic effect due to methanol being a precursor of an intermediate of the formation of higher alcohols.

The addition of specific source alcohols was further found to have the effect of defining the composition of the product alcohol mixture by favouring production of specific higher alcohols over other higher alcohols. Without being bound by theory it is assumed that the formation of higher alcohols proceed mainly by the beta addition of a methyl group to a source alcohol which is faster than the alpha addition in the chain growth of higher alcohols.

Therefore, a mixture of a first source alcohol and a second source alcohol is to be added to the synthesis gas with the objective of forming a product mixture dominated by specific branched product alcohols.

When the first source alcohol is methanol and the second source alcohol is ethanol, the initial product alcohol mixture will be dominated by 1-propanol, as shown by example 3.2 in Table 2.

When the first source alcohol is methanol and the second source alcohol is 1-propanol, the initial product alcohol mixture will be dominated by 2-methyl-1-propanol as shown by example 3.3 in Table 2.

The source alcohols may be admixed in the liquid phase into the synthesis gas upstream the alcohol reactor and evaporate subsequently in the synthesis gas.

The synthesis of higher alcohols is preferably carried out at a pressure above 2 MPa, typically between 2 and 15 MPa and preferably at a temperature above 250° C., preferably between 270° C. and 330° C.

The synthesis of higher alcohols can be performed in an adiabatically operated reactor with quench cooling or preferably in a cooled boiling water reactor producing high pressure steam. In the boiling water reactor large diameter tubes may be used due to a modest reaction rate of higher alcohol formation compared to the reaction rate of methanol formation.

In the disclosed synthesis of higher alcohols small amounts of aldehydes and ketones together with other oxygenates are formed as by-products. These by-products may form azeotropic mixtures with the higher alcohols or have boiling points close to the alcohols and leave the purification of the product difficult. The removal of such oxygenates has not been considered in relation to higher alcohols but is known for methanol formation as disclosed in US application US2006/0235090.

In a specific embodiment of the disclosure, the alcohol product being withdrawn from the alcohol synthesis step is subjected to a hydrogenation step in presence of a hydrogenation catalyst, wherein the oxygenate by-products are hydrogenated to their corresponding alcohols. Thereby, the final distillation of the product is much improved.

For the purpose of the product hydrogenation, the product alcohol mixture being withdrawn from the alcohol synthesis is cooled in a feed effluent heat exchanger to a temperature between 100° C. and 200° C. and introduced into a hydrogenation reactor containing a bed of hydrogenation catalyst. Useful hydrogenation catalysts comprise copper and optionally one or both of zinc oxide and aluminium oxide or as an alternative, the hydrogenation catalyst may comprise platinum and/or palladium.

The thus treated product alcohol mixture is passed to a distillation step, wherein water and a part of the higher alcohols are separated from the remaining higher alcohols. The separated amount of alcohols may be admixed into the optionally purified synthesis gas as described hereinbefore according to the desired end product.

In a preferred embodiment all or a part of the product alcohol mixture is recycled optionally after being passed through a separation such as a distillation step or a chromatographic separation, wherein a separation may take place according to branching and/or chain length.

Separation in combination with recycling provides the possibility to partially or fully recycle only short linear alcohols, such as those comprising less than 4 carbon atoms (methanol, ethanol and propanol), as these alcohols may react further as source alcohols forming desirable product alcohols.

In another preferred embodiment, separation in combination with recycling may be used for partially or fully withdrawing branched alcohols, as they are end-products, which cannot contribute further to the synthesis of higher alcohols and provide recycling of a mixture mainly consisting of non-branched alcohols.

### EXAMPLES

### Example 1

Alkali modified (1 wt. % K) alcohol preparation catalyst consisting of oxides of copper, zinc and aluminium (commercially available from Haldor Topsoe A/S under the trade name “MK-121”) is activated at 1 bar, with a 4000 Nl/h/kg cat space velocity of 3% H2, 0.2% CO, 4.4% CO₂ in N₂ gas mixture starting at 170° C. and heating up to 225° C. with a 10° C./min ramp. It is kept at 225° C. for two hours. The thus activated catalyst consists of metallic copper, zinc oxide and aluminium oxide promoted with potassium carbonate.

The catalyst evaluation experiments were carried out in a copper lined stainless steel plug-flow reactor (19 mm ID) containing catalyst pellets (10-20 g, pellet diameter 6 mm and height 4 mm) held in place by quartz wool.

The reactor effluent was analyzed by on-line gas chromatograph. The liquid composition was identified with a GC-MS.

The reaction temperature, gas composition, alcohol cofeeding, space velocity and pressure effects were evaluated and the results are shown in Tables 1 and 2 below. The synthesis gas mixture contained H₂ and CO (with the specified ratios in the Tables), 2-5 vol. % CO₂ and 3 vol. % Ar.

An increase in temperature with feed of only methanol increases production of 2-methyl-1-propanol. It is believed that with higher temperature the reactions from methanol via ethanol and 1-propanol to 2-methyl-1-propanol are pushed towards secondary reactions forming the higher alcohols, and at the same time that a temperature above 330° C. will deactivate the catalyst.

TABLE 1
Effect of temperature on the higher alcohols production: H2/CO = 1.1,
80 bar, SV = 2000 Nl/kg · cat/h, 20 g catalyst.

| Temperature (° C.) | 280 | 300 | 320 |
|---|---|---|---|
| MeOH (inlet, g/h) | 16.461 | 9.369 | 5.121 |
| CO % conversion | 13 | 22 | 29 |

Exit composition (g/h/g · cat)

| | | | |
|---|---|---|---|
| Methanol | 0.8486 | 0.4681 | 0.2671 |
| Ethanol | 0.0194 | 0.0191 | 0.0120 |
| 1-Propanol | 0.0130 | 0.0206 | 0.0163 |
| 2-Propanol | 0.0009 | 0.0022 | 0.0021 |
| 2-Methyl-1-propanol | 0.0004 | 0.0219 | 0.0303 |
| Other butanols | 0.0047 | 0.0065 | 0.0050 |
| Pentanols | 0.0050 | 0.0111 | 0.0110 |
| Hexanols and higher | 0.0024 | 0.0054 | 0.0061 |
| Total (Ethanol | 0.0458 | 0.0867 | 0.0828 |
| and higher) | | | |

### Experiment 2

The results of examples 3.1, 3.2 and 3.3 presented in Table 2 show that the source alcohols play an important role in defining the product alcohols. When the source alcohol is methanol only as in Example 3.1, the dominant product alcohol is ethanol, with similar levels of production of 1-propanol and 2-methyl-1-propanol.

TABLE 2
Effect of inlet composition on the higher alcohols production: H₂/CO =
0.5, 100 bar, SV = 20000 Nl/kg · cat/h, 10 g catalyst.

Source alcohols

| | | Methanol | Methanol |
|---|---|---|---|
| | Methanol | Ethanol | 1-Propanol |
| Example | 3.1 | 3.2 | 3.3 |
| Methanol (inlet, g/h) | 23.7 | 23.5 | 23.9 |
| Ethanol (inlet, g/h) | — | 112.4 | — |
| 1-Propanol (inlet, g/h) | — | — | 149.6 |
| CO % conversion | 5 | 5 | 0.01 |

Exit composition (g/h/g · cat)

| | | | |
|---|---|---|---|
| Methanol | 0.894 | 1.6934 | 1.4988 |
| Ethanol | 0.0469 | 7.7949 | 0.0000 |
| 1-Propanol | 0.0433 | 0.4484 | 12.3578 |
| 2-Propanol | 0 | 0.0158 | 0.0589 |
| 2-Methyl-1-propanol | 0.0442 | 0.0471 | 0.3760 |
| Other butanols | 0.0123 | 0.4060 | 0.2269 |
| Pentanols | 0.0231 | 0.1013 | 0.1624 |
| Hexanols and higher | 0.0092 | 0.0086 | 0.0765 |
| Total (excluding | 0.179 | 1.0272 | 0.9007 |
| reactant alcohols) | | | |

When the source alcohol is a mixture of methanol and ethanol the major product is 1-propanol as in Example 3.2, but a significant amount of other (linear) butanols is also produced, whereas the production of 2-methyl-1-propanol is a factor 8 below the sum of linear butanols.

When the source alcohol is a mixture of methanol and 1-propanol the major product is 2-methyl-1-propanol as in Example 3.3, which in this case has a concentration which is 1.65 times above the concentration of linear butanols.

This example demonstrates that the selectivity for production of a specific alcohol may be controlled by the choice of source alcohols fed to the reactor.

The results reported in Table 1 demonstrate that when the source alcohol comprises only methanol and conditions are mild. the initial products are ethanol and 1-propanol. With an increased temperature the formed 1-propanol may react further to form 2-methyl-1-propanol.

Table 2 demonstrates that when the source alcohol comprises methanol as well as ethanol the initial product is 1-propanol.

When the source alcohol comprises methanol as well as 1-propanol the initial product is 2-methyl-1-propanol.

With an increased residence time or temperature the formed 1-propanol may react further to form 2-methyl-1-propanol, and in this case the conversion of CO will also be higher.

1. Process for the preparation of a product alcohol mixture comprising the steps of:
(a) providing a synthesis gas comprising carbon monoxide and hydrogen,
(b) providing an amount of methanol and a second source alcohol Rn—CH2-CH2-OH comprising n+2 carbon atoms (Rn=CnH2n+1, n≧0) to the synthesis gas to obtain a selective alcohol synthesis mixture,
(c) converting the selective synthesis mixture in presence of one or more catalysts catalysing the conversion of the synthesis gas mixture into a product alcohol mixture in which the initially dominating alcohol is a preferred Cn+3 alcohol having the structure Rn—CH(CH3)-CH2-OH,
(d) withdrawing the product alcohol mixture of step (c).

2. Process according to claim 1 wherein methanol is present in a concentration corresponding within ±10% from the equilibrium at reaction temperature and the second source alcohol concentration in the selective alcohol synthesis mixture is 0.1-60 volume %.

3. Process according to claim 1, wherein the second source alcohol is ethanol and the preferred alcohol is 1-propanol.

4. Process according to claim 1, wherein the second source alcohol is 1-propanol, and the preferred alcohol is 2-methyl-1-propanol.

5. Process according to claim 1, wherein the preferred alcohol is present in higher concentration than any of the alcohols being isomers to the preferred alcohol.

6. Process of claim 1, wherein the one or more catalysts in step (c) comprise
either copper or copper oxide on a support, or
either copper or copper oxide, and
one or both of zinc oxide and aluminium oxide, and
may optionally be promoted with one or more promoters selected from alkali metals, basic oxides of earth alkali metals and lanthanides, and
in which the copper may be provided as metallic copper or copper oxide.

7. Process of claim 1, wherein metal carbonyl compounds are substantially absent from selective synthesis mixture of step (c), such as having a concentration of 0-10 ppbw, or preferably 0-2 ppbw.

8. Process of claim 7, wherein metal carbonyl compounds are removed from the synthesis gas or the selective synthesis mixture by contacting the synthesis gas or the selective synthesis mixture with a sorbent.

9. Process of claim 1, wherein the conversion of the synthesis gas mixture is performed at a temperature of between 270° C. and 330° C.

10. Process of claim 1, wherein the conversion of the synthesis gas mixture is performed at a pressure of between 2 and 15 MPa.

11. Process of claim 1, comprising the further steps of
(d) cooling the withdrawn product alcohol mixture in step (c); and
(e) contacting the cooled product alcohol mixture with a hydrogenation catalyst.

12. Process according to claim 1, further comprising a step (f) wherein a part of the product alcohol mixture is recycled to be combined with the selective alcohol synthesis mixture of step (b).

13. Process according to claim 1, further comprising a step (g) wherein the product mixture is separated into at least two fractions in a separation step, such as a chromatographic separation or a distillation.

14. Process according to claim 12, further comprising a step (g) wherein the product mixture is separated into at least two fractions in a separation step, such as a chromatographic separation or a distillation., wherein the recycled part of the product mixture is selected from the separated fractions according to branching of the product alcohols.

15. Process according to claim 12, further comprising a step (g) wherein the product mixture is separated into at least two fractions in a separation step, such as a chromatographic separation or a distillation, wherein the recycled part of the product mixture is selected from the separated fractions according to molecular size.
